(19) United States
(12) Patent Application Publication
(10) Pub. No.: US 2021/0154373 A1
(43) Pub. Date: May 27, 2021

(54) METHOD FOR IMMOBILIZING HEPARIN AND NO-GENERATING CATALYST AND CARDIOVASCULAR DEVICE HAVING SURFACE MODIFIED USING THE SAME

(71) Applicant: AJOU UNIVERSITY INDUSTRY-ACADEMIC COOPERATION FOUNDATION, Gyeonggi-do (KR)

(72) Inventors: Ki Dong PARK, Seoul (KR); Le Thi PHUONG, Ho Chi Minh City (VN); Tran Dieu LINH, Bien Hoa City (VN)

(73) Assignee: AJOU UNIVERSITY INDUSTRY-ACADEMIC COOPERATION FOUNDATION, Gyeonggi-do (KR)

(21) Appl. No.: 16/952,627

(22) Filed: Nov. 19, 2020

(30) Foreign Application Priority Data
Nov. 21, 2019 (KR) 10-2019-0150551

(51) Int. Cl. A61L 31/16; A61L 31/14; A61L 31/02

(52) U.S. Cl. CPC A61L 31/16; A61L 31/148; C08L 5/10; A61L 31/026; A61L 31/022

(57) ABSTRACT

The present disclosure relates to a method for immobilizing heparin and a NO-generating catalyst and a cardiovascular device having a surface modified using the same, and more particularly, to a method of co-immobilizing a heparin-phenol derivative and copper nanoparticles as a NO-generating catalyst on the surface of a material by a polyphenol oxidase-mediated reaction, a material having a surface with heparin and a NO-generating catalyst co-immobilized thereon by using the method, and a cardiovascular device including the material. It has been confirmed that a surface having heparin and the NO-generating catalyst co-immobilized thereon by the method of the present disclosure has high in vivo stability, continuously generates NO, and also promotes the proliferation of endothelial cells while significantly inhibiting the adhesion and activation of platelets and smooth muscle cells. Thus, the method may be advantageously applied to cardiovascular devices for inhibiting thrombosis and restenosis.

### BACKGROUND

### 1. Technical Field

The present disclosure relates to a method for immobilizing heparin and a NO-generating catalyst and a cardiovascular device having a surface modified using the same, and more particularly, to a method of co-immobilizing a heparin-phenol derivative and copper nanoparticles as a NO-generating catalyst on the surface of a material by a polyphenol oxidase-mediated reaction, a material having the surface on which heparin and a NO-generating catalyst are co-immobilized by using the method, and a cardiovascular device including the material.

### 2. Related Art

Cardiovascular diseases (CVDs) are a major cause of death worldwide. According to the 2017 reports of the American College of Cardiology and the European Heart Network, CVDs caused 800 thousand deaths and 3.9 million deaths in the United States and Europe, respectively. Although the cardiovascular device market has developed due to rapidly increasing cardiovascular disease patients, innovative technologies for modifying cardiovascular devices capable of improving adaptability and effectiveness for long-term clinical applications remain insufficient.

In general, it is known that nitric oxide produced from endothelial cells in a healthy vascular system prevents thrombus formation by inhibiting the adhesion and activation of platelets. However, if endothelial cells in an region implanted with a cardiovascular device are damaged, normal nitric oxide production is stopped, resulting in thrombus formation, and then restenosis occurs due to the migration and proliferation of smooth muscle cells.

To solve this problem, many studies have been conducted to control the migration of platelets, endothelial cells and smooth muscle cells by modifying the surface of cardiovascular devices using bioactive molecules or drugs, such as vascular endothelial growth factor (VEGF), paclitaxel, phosphorylcholine, albumin, heparin, and NO-releasing molecules (S. L. Chin-Quee et al., Biomaterials, 31:648-657, 2010; D. Trabattoni et al., Int J Cardiol, 135:e65-6, 2009).

Heparin is a chemical analogue of heparan sulfate proteoglycan which provides anticoagulant activity to vascular endothelium. Heparin is an anticoagulant which is most commonly used. Since heparin has excellent anticoagulant activity, it is widely used for surface modification of blood-contacting devices such as catheters, vascular grafts, coronary stents, and cardiopulmonary bypass machines. In order to immobilize heparin on the surface of these devices, various methods have been applied, such as blending, surface coating, grafting and layer-by-layer assembly, because heparin has a strong negative charge, is rich in carboxyl groups and sulfo groups, and has other various functional groups on the backbone thereof (P. Le Thi et al., ACS Appl Mater Interfaces, 9:20376-20384, 2017; Y. K. Joung et al., Colloids Surf B Biointerfaces, 99:102-107, 2012).

In addition, nitric oxide (NO) is a gaseous molecule synthesized by the endothelium and is known to play an important role in the cardiovascular system. Nitric oxide functions to reduce platelet adhesion and activation, inhibit smooth muscle cell growth, promote endothelial cell proliferation and inhibit microbial growth. In general, current NO-based substances for cardiovascular treatment can be classified into: substances that actively release NO and NO analogs; and substances that stimulate the endogenous enzymatic production of NO. The strategy has problems such as short half-life (2 to 5 seconds), passive release of NO during production, instability during storage and sterilization, and release of toxic by-products.

In order to solve the above problems, strategies focused on the decomposition of endogenous S-nitrosothiols (RSNOs) for releasing NO have recently attracted much attention. Among them, NO generated from transition metal ion catalysts (Cu²⁺, Hg²⁺, Fe²⁺, Ag⁺, and organic cesium, etc.) offers distinct advantages over active release strategies, including maintenance of NO activity, protection from free radicals, and reduced cytotoxicity levels.

Accordingly, the present inventors have made extensive efforts to develop a method of co-immobilizing heparin and a NO-generating catalyst in order to improve the antithrombotic and anti-restenotic properties of a blood-contacting device. As a result, the present inventors have developed a method of co-immobilizing a heparin-phenol derivative (HT) and copper nanoparticles as a NO-generating catalyst on the surface of a material by a tyrosinase-mediated reaction, and have found that the heparin/CuNP-immobilized surface of the material surface-modified by the method significantly inhibits the adhesion and activation of platelets and smooth muscle cells (SMCs) in the presence of a NO donor, but promotes the proliferation of endothelial cells, thereby completing the present disclosure.

### SUMMARY

An object of the present disclosure is to provide a method for co-immobilizing heparin and a NO-generating catalyst.

Another object of the present disclosure is to provide either a material modified using the above method or a cardiovascular device for inhibiting thrombosis or restenosis.

To achieve the above objects, the present disclosure provides a method for co-immobilizing heparin and a NO-generating catalyst, the method including steps of: (a) producing a heparin-phenol derivative by reacting a functional group-activated heparin with a phenol derivative; and (b) treating the surface of a material with a mixture solution containing the heparin-phenol derivative, polyphenol oxidase and a NO-generating catalyst.

In one preferred embodiment of the present disclosure, the phenol derivative may be one or more selected from the group consisting of tyramine, tyrosine hydroxyphenyl propionic acid, and 4-hydroxyphenyl acetic acid.

In another preferred embodiment of the present disclosure, the functional group-activated heparin in step (a) may be a carboxyl group-activated heparin, a hydroxyl group-activated heparin, or an aldehyde group-activated heparin.

In still another preferred embodiment of the present disclosure, the carboxyl group-activated heparin may be produced by reacting heparin with 1-ethyl-3-(3-dimethylaminopropyl)carbodiimide (EDC) and N-hydroxysuccinimide (NHS), the hydroxyl group-activated heparin may be produced by reacting heparin with N,N′-dicyclohexylcarbodiimide and 4-dimethylaminopiridine, and the aldehyde group-activated heparin may be produced by oxidizing heparin with sodium periodate (NaIO₄).

In yet another preferred embodiment of the present disclosure, the polyphenol oxidase in step (b) may be tyrosinase.

In still yet another preferred embodiment of the present disclosure, the heparin-phenol derivative in step (b) may be contained in an amount of 0.5 to 1 wt %, the polyphenol oxidase may be contained in an amount of 0.1 to 0.4 kU/ml, and the NO-generating catalyst may be contained in an amount of 0.2 to 1 mg/ml.

In a further preferred embodiment of the present disclosure, the treating of the surface of the material in step (b) may be performed at a pH of 8.0 to 9.0 for 1 to 5 hours.

In another further preferred embodiment of the present disclosure, the mixture solution in step (b) may further contain calcium peroxide.

In still another further preferred embodiment of the present disclosure, the material in step (b) may be a biocompatible material selected from among a biodegradable polymer, a non-degradable polymer, a metal and a ceramic material.

In yet another further preferred embodiment of the present disclosure, the biodegradable polymer may be one or more selected from the group consisting of polyglycolic acid, polylactide, poly(ε-caprolactone), poly(lactide-co-glycolide), poly(lactide-co-ε-caprolactone), poly(ε-caprolactone-co-glycolide), poly(ε-caprolactone-co-glycolide-co-lactide), polyhydroxybutyrate valerate (PHBV), polyorthoester (POE), polyethyleneoxide/polybutylene terephthalate (PEO/PBTP), chitosan, and collagen, and the non-degradable polymer may be one or more selected from the group consisting of polyvinyl chloride, polyurethane (PUR), polyethylene terephthalate (PETP), polyamides, polyester, polyester elastomers, polymethylmethacrylate, polyolefins, silicone, polytetrafluoroethylene (PTFE), and polyfluorocarbon.

The metal may be one or more selected from the group consisting of stainless steel, cobalt, chromium, titanium, nitinol, tantalum, and alloys thereof.

The ceramic material may be one or more selected from the group consisting of calcium phosphate, alumina, zirconia, silicon nitride, crystallized glass, and carbon materials.

To achieve the other object, the present disclosure provides a material for inhibiting thrombosis and restenosis, the material having a surface with heparin and a NO-generating catalyst co-immobilized thereon by the above-described method.

The present disclosure also provides a cardiovascular device for inhibiting thrombosis and restenosis, the cardiovascular device including a material having a surface with heparin and a NO-generating catalyst co-immobilized thereon by the above-described method.

According to a preferred embodiment of the present disclosure, the cardiovascular device may be a stent.

### BRIEF DESCRIPTION OF THE DRAWINGS

FIG. 1 is a schematic view showing that surfaces having heparin and copper particles introduced thereto are produced by immobilizing a heparin-phenol derivative and copper ions on various surfaces using polyphenol oxidase, and that the produced surfaces inhibit platelet activity, inhibit smooth muscle cell proliferation, and activate endothelial cell proliferation.

FIG. 2 shows the results of ¹H NMR spectrum analysis of a carboxyl group-activated heparin-phenol derivative (FIG. 2a), a hydroxyl group-activated heparin-phenol derivative (FIG. 2b), and an aldehyde group-activated heparin-phenol derivative (FIG. 2c).

FIG. 3 shows the results of analyzing the heparin amounts of surfaces having heparin and copper particles introduced thereto (FIG. 3a), surface stability of the surfaces (FIG. 3b), the degree of hydrophilicity of the surfaces (FIG. 3c), and changes in surface elements by surface modification (FIGS. 3d and 3e).

FIG. 4 shows the results of measuring nitric oxide release behaviors of surfaces having heparin and copper particles introduced thereto (FIG. 4a), and the amount of copper released from the surfaces (FIG. 4b).

FIG. 5 is a schematic view showing a method of immobilizing heparin using the catalytic reaction of tyrosinase and calcium peroxide.

FIG. 6 shows the results of measuring changes in the introduced amount of heparin depending on the calcium peroxide concentration and the reaction time in the method of immobilizing heparin using the catalytic reaction of tyrosinase and calcium peroxide.

FIG. 7 shows the results of water contact angle analysis performed to examine surface stability depending on the reaction time in the method of immobilizing heparin using the catalytic reaction of tyrosinase and calcium peroxide.

FIG. 8 shows the results of analyzing the adsorption and activation of platelets on surfaces having heparin and copper particles introduced thereto (FIG. 8a), and the intracellular levels of cyclic GMP synthesized by platelets (FIG. 8b).

FIG. 9 shows the results of measuring the viability of vascular endothelial cells on surfaces having heparin and copper particles introduced thereto (FIGS. 9a and 9b), and cell migration rates on the surfaces (FIGS. 9c and 9d).

FIG. 10 shows the inhibition of proliferation and activity of smooth muscle cells on surfaces having heparin and copper particles introduced thereto.

FIG. 11 is a schematic view showing a method of immobilizing heparin and a NO-generating catalyst depending on the kind of material surface.

FIG. 12 shows the results of analyzing the heparin amounts of various material surfaces having heparin and copper particles introduced thereto, depending on the kind of material surface.

FIG. 13 shows the results of measuring the nitric oxide release behaviors of various material surfaces having heparin and copper particles introduced thereto, depending on the kind of material surface.

FIG. 14 shows the results of analyzing the degrees of proliferation and adhesion of endothelial cells depending on the kind of material surface.

FIG. 15 shows the results of analyzing the degrees of proliferation and adhesion of smooth muscle cells depending on the kind of material surface.

### DETAILED DESCRIPTION

Hereinafter, the present disclosure will be described in detail.

In one aspect, the present disclosure is directed to a method for co-immobilizing heparin and a NO-generating catalyst, the method including steps of: (a) producing a heparin-phenol derivative by reacting a functional group-activated heparin with a phenol derivative; and (b) treating the surface of a material with a mixture solution containing the heparin-phenol derivative, polyphenol oxidase and a NO-generating catalyst.

In another aspect, the present disclosure is directed to a composition for co-immobilizing heparin and a NO-generating catalyst, the composition containing a heparin-phenol derivative, polyphenol oxidase, and a NO-generating catalyst.

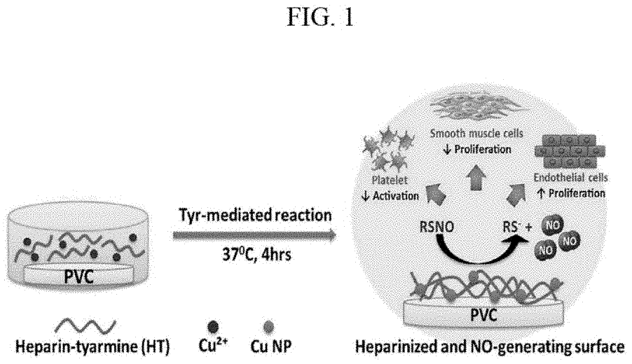

The present inventors have developed a method of co-immobilizing heparin and a NO-generating catalyst in order to improve the thrombosis or restenosis inhibitory effect of a blood-contacting device such as a cardiovascular device. Specifically, as schematically shown in FIG. 1, the present inventors have co-immobilized a heparin-phenol derivative (heparin-tyramine derivative; HT) and copper nanoparticles as a NO-generating catalyst on the surface of a material by a “one-pot” tyrosinase-mediated reaction. Under aerobic conditions, tyrosinase can provide binding affinity for a variety of substrates, including metal ions, organic substances and inorganic surfaces, by oxidizing the phenol moiety of the heparin-phenol derivative into catechol and O-quinone moieties. As a result, a metal-catechol coordination polymer network is formed, so that the material surface is coated with an HT/Cu mixture.

In the present disclosure, the phenol derivative may be one or more selected from the group consisting of tyramine, tyrosine hydroxyphenyl propionic acid, and 4-hydroxyphenyl acetic acid. Preferably, the phenol derivative may be tyramine.

In the present disclosure, the functional group-activated heparin in step (a) may be a carboxyl group-activated heparin, a hydroxyl group-activated heparin, or an aldehyde group-activated heparin. Preferably, it may be a carboxyl group-activated heparin.

Specifically, the carboxyl group-activated heparin may be produced by reacting heparin with 1-ethyl-3-(3-dimethylaminopropyl) carbodiimide (EDC) and N-hydroxysuccinimide (NHS), the hydroxyl group-activated heparin may be produced by reacting heparin with N, N′-dicyclohexylcarbodiimide and 4-dimethylaminopiridine, and the aldehyde group-activated heparin may be produced by oxidizing heparin with sodium periodate (NaIO₄).

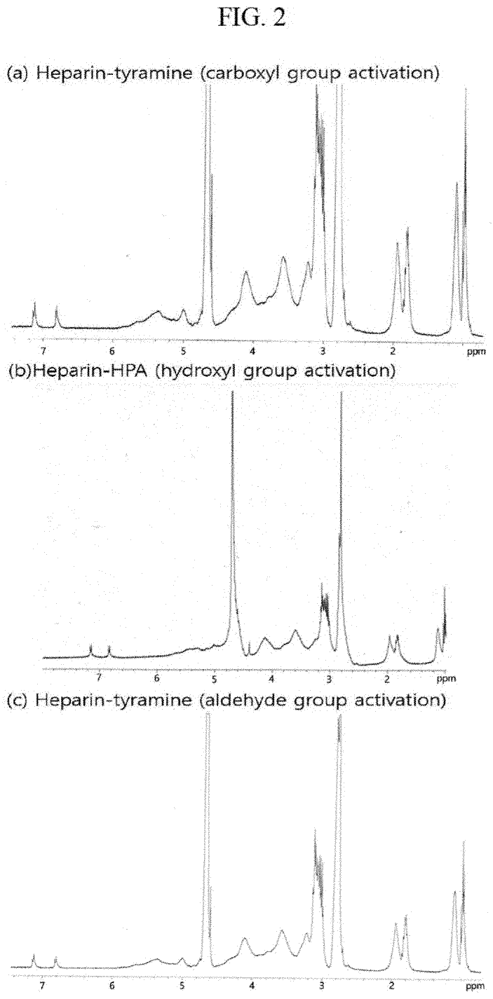

In one specific example of the present disclosure, synthesis of the carboxyl group-activated heparin, the hydroxyl group-activated heparin and the aldehyde group-activated heparin was performed as described above. As shown in FIG. 2, it was confirmed through 1H NMR spectrum analysis that the synthesis was well performed.

In the present disclosure, the NO-generating catalyst in step (b) may be one or more selected from the group consisting of copper ion, mercury ion, iron ion, silver ion, and organic cesium. Preferably, it may be copper ion.

In addition, a NO donor that may be applied to the present disclosure may be one or more selected from the group consisting of S-nitroso-N-acetylpenicillamine (SNAP), N-diazeniumdiolates (NONOates), S-nitrosocysteine, and S-nitrosoalbumin.

In the present disclosure, the polyphenol oxidase in step (b) may be tyrosinase.

In the present disclosure, in step (b), the surface of the material may be treated with a solution obtained by mixing 0.5 to 1 wt % of the heparin-phenol derivative, 0.1 to 0.4 kU/ml of polyphenol oxidase and 0.2 to 1 mg/ml of the NO-generating catalyst, and the treatment of the surface may be performed at a pH of 8.0 to 9.0 for 1 to 5 hours.

In the present disclosure, the material in step (b) may be a biocompatible material selected from among a biodegradable polymer, a non-degradable polymer, a metal and a ceramic material, but is not limited thereto and any material may be used without limitation as long as it is a material that is applied for cardiovascular devices for inhibiting thrombosis and restenosis.

The biodegradable polymer may be one or more selected from the group consisting of polyglycolic acid, polylactide, poly(ε-caprolactone), poly(lactide-co-glycolide), poly(lactide-co-ε-caprolactone), poly(ε-caprolactone-co-glycolide), poly(ε-caprolactone-co-glycolide-co-lactide), polyhydroxybutyrate valerate (PHBV), polyorthoester (POE), polyethyleneoxide/polybutylene terephthalate (PEO/PBTP), chitosan, and collagen.

The non-degradable polymer may be one or more selected from the group consisting of polyvinyl chloride, polyurethane (PUR), polyethylene terephthalate (PETP), polyamides, polyester, polyester elastomers, polymethylmethacrylate, polyolefins, silicone, polytetrafluoroethylene (PTFE), and polyfluorocarbon.

The metal may be one or more selected from the group consisting of stainless steel, cobalt, chromium, titanium, nitinol, tantalum, and alloys thereof.

The ceramic material may be one or more selected from the group consisting of calcium phosphate, alumina, zirconia, silicon nitride, crystallized glass, and carbon materials.

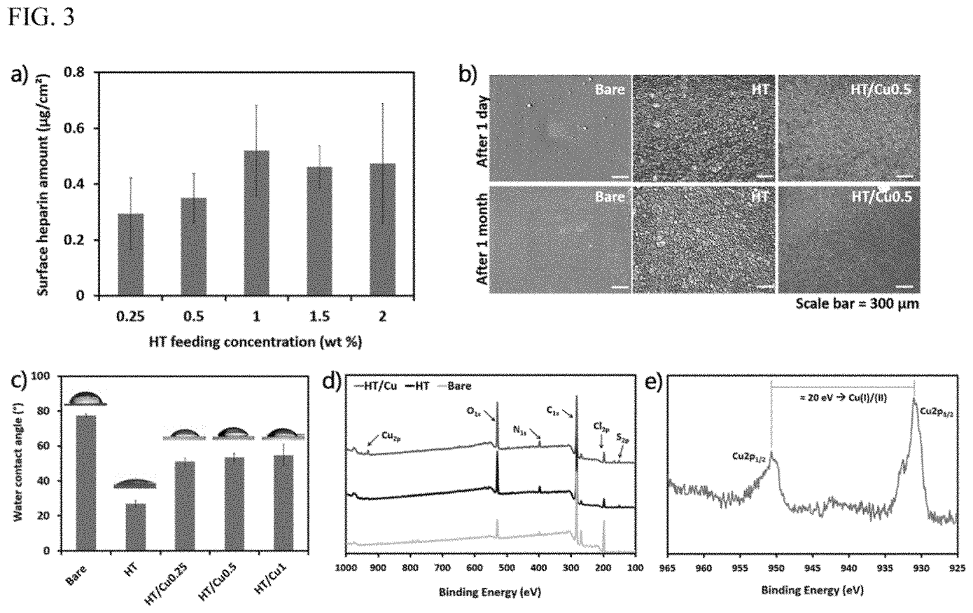

In one specific example of the present disclosure, heparin and copper nanoparticles were co-immobilized on a PVC surface by a tyrosinase-mediated reaction. As a result of analyzing the heparin amount of the heparin-immobilized surface, it was confirmed that the amount of heparin immobilized on the surface increased depending on the HT feeding concentration (FIG. 3a), and reached a stable value of 0.58 μg/cm² at 1 wt % of HT. Thus, in the present disclosure, heparin and copper nanoparticles were co-immobilized on the PVC surface by using 1 wt % of HT and varying concentrations (0.5, 0.75 and 1 mg/ml) of CuSO₄ through the catalytic activity of tyrosinase (0.4 kU/ml).

As a result of analyzing the HT/Cu surfaces with heparin and cupper nanoparticles co-immobilized thereon, it was confirmed that deposition of many spherical copper nanoparticles was observed and all the surfaces after immobilization were stably maintained even after they were immersed in a PBS solution for 30 days (FIG. 3b).

As a result of analyzing the wettability of each of the surfaces by water contact angle measurement, it was confirmed that the bare PVC surface was hydrophobic and had a water contact angle (WCA) of 78°. In addition, it was confirmed that the WCA of the HT surface after HT immobilization decreased to 23° due to introduction of hydrophilic sulfate and hydroxyl group-activated heparin, but the WCA of the HT/Cu surface with copper nanoparticles deposited thereon increased to 45° due to the hydrophobicity of copper (FIG. 3c). In addition, as a result of measuring the coating thickness of the HT/Cu surface, it was confirmed that the coating thickness of the HT/Cu surface increased gradually as the CuSO₄ feeding concentration increased (Table 1).

Furthermore, through the spectrum analysis of HT/Cu (FIGS. 3d and 3e), it was confirmed that heparin and copper nanoparticles were successfully co-immobilized on the PVC surface by the “one-pot” tyrosinase-mediated reaction.

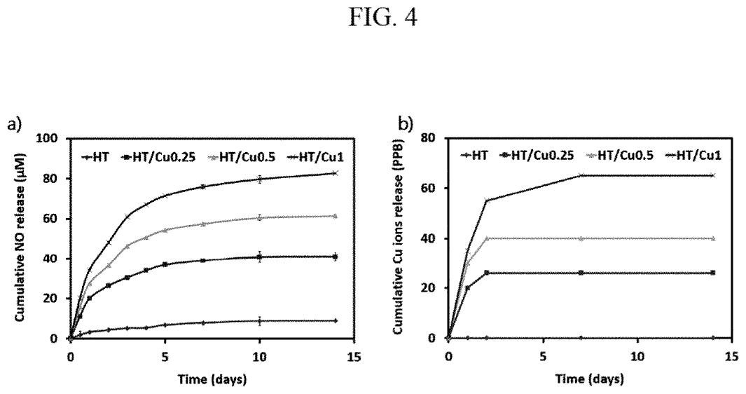

In another specific example of the present disclosure, analysis was performed on whether NO is effectively released from the surfaces with heparin and copper nanoparticles co-immobilized thereon. As shown in FIG. 4a, the release rate of NO from the HT/Cu surface significantly increased in the presence of GSNO, and was maintained for 14 days. In addition, the release of NO was proportional to the feeding concentration of CuSO₄ used for immobilization. Specifically, it was confirmed that the cumulative release of NO after 14 days was 80 μM for HT/Cu1, 58 μM for HT/Cu0.5, and 32 μM for HT/Cu0.25.

In addition, as a result of analyzing the amount of copper released, as shown in FIG. 4b, it was confirmed that the HT/Cu surface showed a high release rate for the first 7 days, and then showed a low release rate, and the the HT/Cu surface was stably maintained even after it was immersed in PBS for 7 days. In addition, it was confirmed that, as the CuSO₄ concentration increased, the amount of copper nanoparticles immobilized increased and the amount of Cu ions released increased.

In the present disclosure, the solution in step (b) may further contain calcium peroxide. The solution in step (b) may further contain calcium peroxide in an amount of preferably 0.025 wt % to 0.2 wt %, more preferably 0.05 wt % to 0.1 wt %.

In still another specific example of the present disclosure, the catalytic reaction of polyphenol oxidase and calcium peroxide was used to more effectively immobilize heparin on a surface.

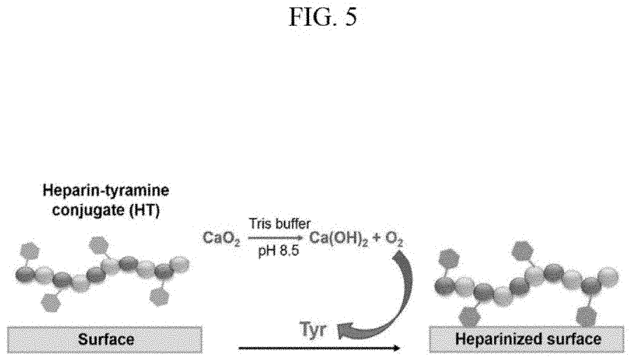

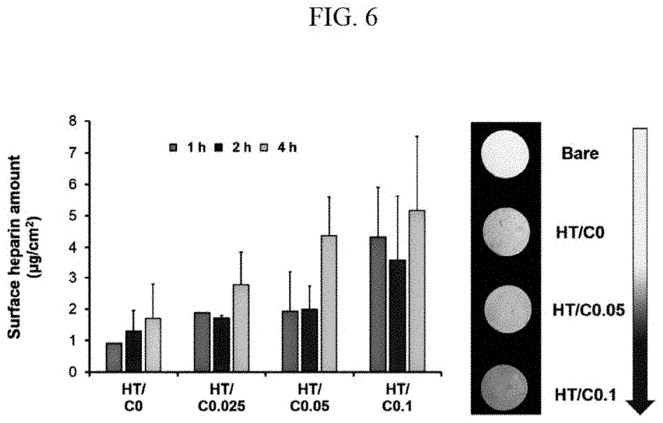

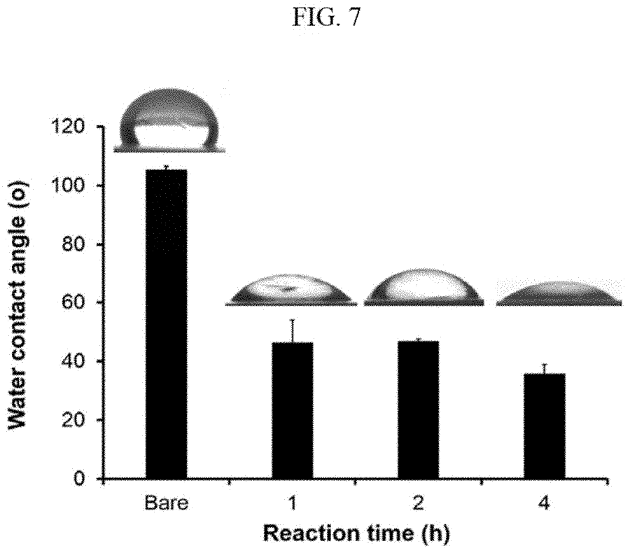

As schematically shown in FIG. 5, the tyrosinase reaction is promoted by calcium peroxide, so that heparin is effectively immobilized on the surface. As a result of analyzing the amount of heparin immobilized and the water contact angle depending on the calcium peroxide concentration and the reaction time, as shown in FIG. 6, it was confirmed, as the reaction time increased and the CaO₂ concentration increased, the amount of heparin immobilized increased. As shown in FIG. 7, it was confirmed that, as the reaction time increased, the amount of heparin immobilized on the surface increased, so that the water contact angle of the surface decreased.

In another aspect, the present disclosure is directed to a material having a surface with heparin and a NO-generating catalyst co-immobilized thereon by the method of the present disclosure.

The material may be a biocompatible material selected from among a biodegradable polymer, a non-degradable polymer, a metal and a ceramic material, but is not limited thereto and any material may be used without limitation as long as it is a material that is applied for cardiovascular devices for inhibiting thrombosis and restenosis.

In still another aspect, the present disclosure is directed to a cardiovascular device for inhibiting thrombosis and restenosis, the cardiovascular device including a material having a surface with heparin and a No-generating catalyst co-immobilized thereon by the method of the present disclosure.

In the present disclosure, the cardiovascular device may be a blood-contacting device such as a stent, a graft, a stent-graft, a catheter, a heart valve, a filter or tube. Preferably, the cardiovascular device may be a stent.

Platelet activation and aggregation after cardiovascular device implantation are important risk factors for life-threatening restenosis and thrombosis. Therefore, it is very important to design a cardiovascular device having the ability to reduce the adhesion and activation of platelets.

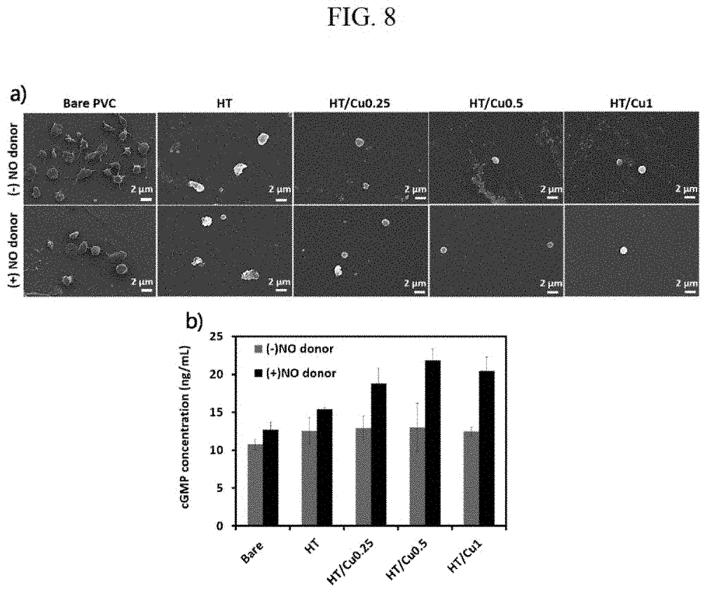

In one specific example of the present disclosure, as a result of observing the morphology of platelets adhering to the HT/Cu surface, it was confirmed that the HT/Cu-immobilized surface showed the lowest platelet adhesion and activation in the presence of a NO donor (FIG. 8a). This result is due to the synergistic effect of heparin and the in situ generation of NO from the surface.

In healthy blood vessels, endothelial cells release NO, stimulate the activity of soluble guanylate cyclase which is a NO acceptor, increase the level of cGMP that controls platelet inhibition, and also induce smooth muscle cell relaxation. Thus, in the present disclosure, the degree of cGMP synthesis in platelets incubated with each of a bare surface and surfaces having HT or HT/Cu immobilized thereon was evaluated. As shown in FIG. 8b, it was confirmed that the HT/Cu surfaces significantly promoted cGMP synthesis in the presence of GSNO, compared to the bare PVC surface and the HT surface. This result means that the HT/Cu surface exhibits the effect of improving plasma formation by producing NO from GSNO and inhibiting the adhesion and activation of platelets.

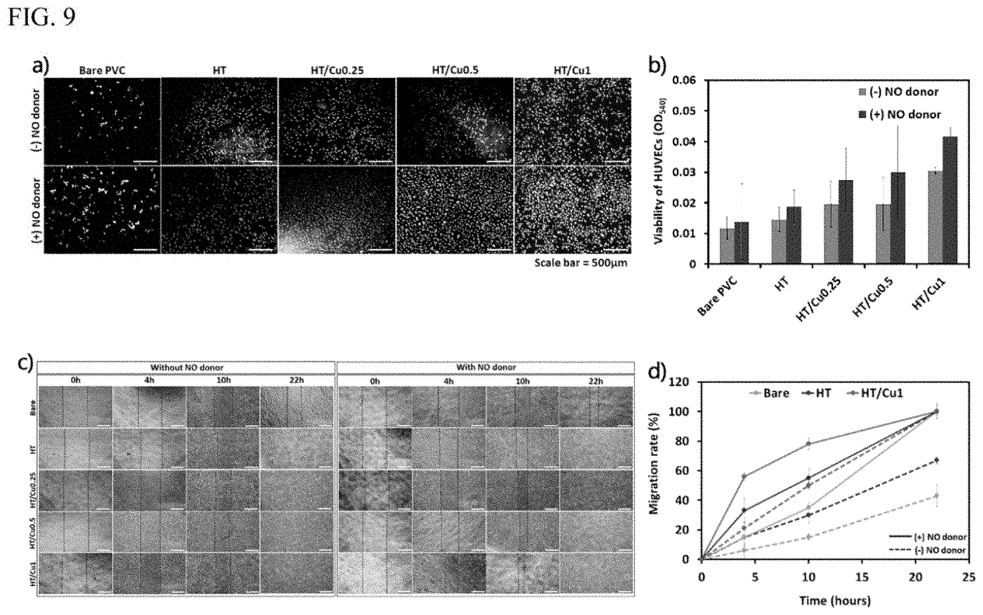

In the present disclosure, the effect of the HT/Cu surface on adhesion and proliferation of endothelial cells was analyzed. It was confirmed that the viability of HUVECs was significantly higher on the HT- or HT/Cu-immobilized surfaces than the bare PVC surface, and particularly, as the amount of CuSO₄ used in the immobilization step in the presence of the NO donor increased, the viability of HUVECs was higher (FIGS. 9a and 9b).

In addition, as a result of analyzing the effect of generated NO on the promotion of endothelial cell by scratch assay, it was confirmed that the migration rate of cells incubated with the heparin-immobilized surface increased compared to the migration rate of cells incubated with the bare PVC surface, and that the cell migration on the HT/Cu surface in the presence of the NO donor significantly increased (FIG. 9c).

Endothelial cells and smooth muscle cells are the two major cell types of blood vessels, each of which performs a separate function to maintain vascular hemostasis. Improved proliferation of endothelial cells reduces the risk of thrombosis and restenosis, whereas proliferation and migration of smooth muscle cells cause luminal narrowing and vascular dysfunction.

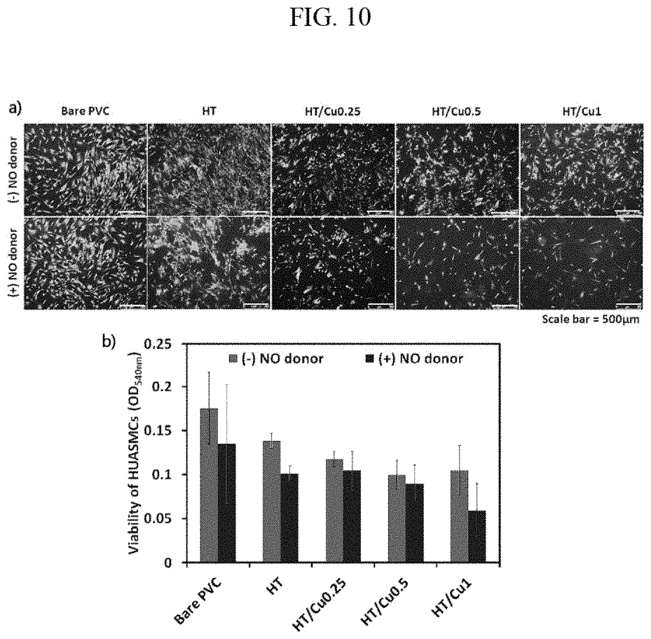

In the present disclosure, as a result of analyzing the effect of the HT/Cu surface on adhesion and proliferation of smooth muscle cells, it was confirmed that, in the absence of the NO donor, there was no significant difference in cell viability between the HT- or HT/Cu-immobilized surface and the bare surface, whereas, in the presence of the NO donor, the HT/Cu surface showed a decrease in the viability of HUASMCs and distinct shrinkage compared to the bare surface and the HT surface (FIG. 10).

That is, it was confirmed that the material surface or cardiovascular device, which has heparin and the NO-generating catalyst co-immobilized thereon, selectively enhanced the proliferation and migration of endothelial cells while significantly inhibiting thrombosis and the adhesion and proliferation of platelets and smooth muscle cells.

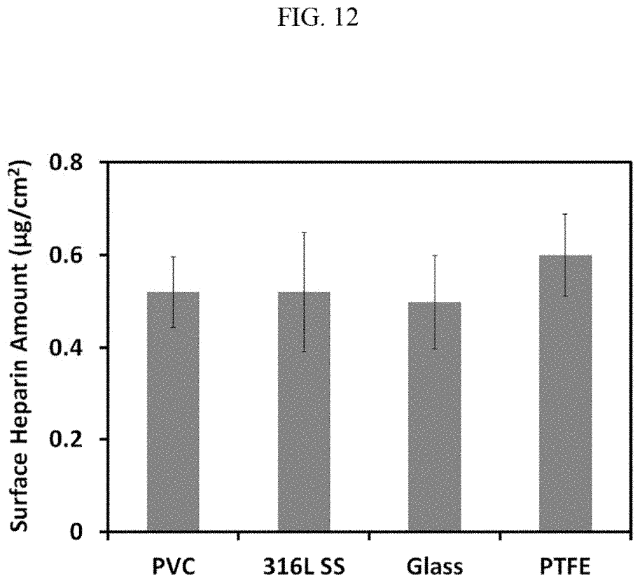

Furthermore, in the present disclosure, analysis was performed on whether heparin and the NO-generating catalyst are effectively co-immobilized regardless of the kind of material surface. As a result of co-immobilizing heparin and copper particles on material surfaces, including polyvinyl chloride (PVC), 316L stainless steel (316L SS), glass, and polytetrafluoroethylene (PTFE), as shown in FIGS. 12 to 15, it was confirmed that the method of the present disclosure efficiently co-immobilized heparin and the NO-generating catalyst on the surfaces regardless of the kind of material surface, and also promoted the proliferation of endothelial cells while significantly inhibiting the adhesion and proliferation of smooth muscle cells.

Hereinafter, the present disclosure will be described in more detail with reference to examples.

These examples are merely to illustrate the present disclosure, and it will be obvious to those of ordinary skill in the art that the scope of the present disclosure is not construed as being limited by these examples.

### [Example 1] Synthesis of Heparin-Phenol Derivatives and Production of Surface Having Heparin and Copper Particles Immobilized Thereon

1-1: Synthesis of Heparin-Phenol Derivatives

<Synthesis of Carboxyl Group-Activated Heparin-Phenol Derivative>

1 g of heparin (heparin sodium salt, Sigma Aldrich, USA) was dissolved in 100 ml of double-distilled water, and then the heparin solution was reacted with 8 mmol of 1-ethyl-3-(3-dimethylaminopropyl)-carbodiimide (EDC) and 5 mmol of N-hydroxysuccinimide (NHS) to activate the carboxyl group of the heparin. Thereafter, the reaction solution was adjusted to a pH of 4.5 to 5 using 5M hydrochloric acid, and then allowed to react for 1 hour. Next, 3 mmol of tyramine hydrochloride was added to the reaction solution, followed by reaction for 24 hours.

After completion of the reaction, the reaction solution was filtered using a syringe filter (450 nm), and then dialyzed with a membrane (3,500-Da molecular weight cut-off) in distilled water. After completion of the dialysis, the solution was freeze-dried to obtain a heparin-phenol derivative.

Through ¹H NMR spectrum analysis, it was confirmed that the heparin-phenol derivative was well synthesized (FIG. 2(a)).

<Synthesis of Hydroxyl Group-Activated Heparin-Phenol Derivative>

1 g of heparin was dissolved in 100 ml of dimethylformamide, and then the heparin solution was reacted with 8 mmol of N,N′-dicyclohexylcarbodiimide and 8 mmol of 4-dimethylaminopiridine to activate the hydroxyl group of the heparin. Thereafter, 3 mmol of hydroxyphenyl propionic acid (replaceable with hydroxyphenyl acetic acid) was added to the reaction solution, followed by reaction for 24 hours.

After completion of the reaction, the reaction solution was filtered through filter paper, and then dialyzed with a membrane (3,500-Da molecular weight cut-off). After completion of the dialysis, the solution was freeze-dried to obtain a heparin-phenol derivative.

Through ¹H NMR spectrum analysis, it was confirmed that the heparin-phenol derivative was well synthesized (FIG. 2(b)).

<Synthesis of Aldehyde Group-Activated Heparin-Phenol Derivative>

1 g of heparin was dissolved in 200 ml of double-distilled water, and then 2.3 mmol of sodium periodate (NaIO₄) was added to the solution. The reaction solution was adjusted to a pH of 5 using hydrochloric acid, and then allowed to react at room temperature for 24 hours under a light-shielded condition. After completion of the reaction, the reaction solution was dialyzed with a membrane (12,000 to 14,000-Da molecular weight cut-off). After completion of the dialysis, the solution was freeze-dried to obtain an aldehyde group-activated heparin.

1 g of the aldehyde group-activated heparin was dissolved in 200 ml of double-distilled water, and then 3 mmol of tyramine hydrochloride was added to the solution, followed by reaction at room temperature for 24 hours under a nitrogen atmosphere. The reaction solution was precipitated in cold ethanol, and then dried, and the precipitate was collected.

Through ¹H NMR spectrum analysis, it was confirmed that the heparin-phenol derivative was well synthesized (FIG. 2(c)).

1-2: Production of Surface Having Heparin and Copper Particles Immobilized Thereon

Mixture solutions were prepared by adding 1 wt % of the carboxyl group-activated heparin-phenol derivative produced in Example 1-1, 0.4 kU/ml of polyphenol oxidase (mushroom-derived tyrosinase; 3,610 units/mg solid, Sigma Aldrich, USA) and 0.25, 0.5 or 1 mg/ml of copper sulfate (CuSO₄) to 0.1M Tris buffer (pH 8). A prepared PVC surface was immersed in each of the prepared mixture solutions, and then allowed to react at 37° C. for 4 hours. After completion of the reaction, each PVC surface was washed with 1 wt % of SDS solution and distilled water, and then dried under a nitrogen gas atmosphere.

In addition, for comparison with the surface (HT/Cu surface) having heparin and copper particles immobilized thereon, a surface (HT surface) having heparin immobilized thereon alone was produced in the same manner as described. As a negative control, an untreated PVC surface (bare PVC surface) was used.

### [Example 2] Analysis of Surface Having Heparin and Copper Particles Introduced Thereto

2-1: Analysis of Amount of Heparin Immobilized

The amount of heparin immobilized on the surface was evaluated using TBO assay. First, 0.0025 wt % of toluidine blue O (TBO) solution was prepared using distilled water. To each of the HT surface, HT/Cu surface and PVC surface produced in Example 1-2, 1 ml of phosphate buffer saline (PBS, 0.01 M, pH 7.4), and 1 ml of the TBO solution was added thereto, followed by incubation at 37° C. for 1 hour. Then, 2 ml of n-hexane was added to each of the solutions, followed by shaking for 5 minutes. After completion of the reaction, 200 μL of each solution was transferred to each well of a 96-well plate, and the absorbance at 620 nm was measured. For measurement, the heparin concentration for the standard curve was set to 0 to 10 μg/ml.

As a result, as shown in FIG. 3a, it was confirmed that the surface heparin amount increased with increasing HT feeding concentration, and reached a stable value of 0.58 μg/cm² at 1 wt % of HT. Thus, immobilization on the PVC surface was performed using 1 wt % of HT and varying concentrations (0.5, 0.75 and 1 mg/ml) of CuSO₄ through the catalytic activity of tyrosinase (0.4 kU/ml).

2-2: Analysis of HT- or HT/Cu-Immobilized Surface

Each of the HT surface, HT/Cu surfaces and PVC surface produced in Example 1-2 was immersed in PBS for 30 days, and then the morphology of each immobilized surface was analyzed by a field emission-scanning electron microscope (FE-SEM).

As a result, as shown in FIG. 3b, it was observed that, unlike the smooth surface of the bare PVC, each of the HT and HT/Cu surfaces was roughly coated. In particular, it was confirmed that deposition of many spherical copper nanoparticles was observed on the HT/Cu surfaces, and all the immobilized surfaces were stably maintained even after they were immersed in the PBS solution for 30 days.

2-3: Measurement of Water Contact Angle of Immobilized Surface

The wettability of each surface before and after immobilization was evaluated using a water contact angle (WCA) measurement system equipped with a camera. 5 μL of a water droplet was dropped onto three different positions of each surface, and the static contact angle of each surface was measured.

As shown in FIG. 3c, it was confirmed that the bare PVC surface was hydrophobic and had a water contact angle of 78°, and that the WCA of the HT surface after HT immobilization decreased to 23° due to introduction of hydrophilic sulfate and hydroxyl group-activated heparin, but the WCA of the HT/Cu surface deposited with copper nanoparticles increased to 45° due to the hydrophobicity of copper.

2-4: Measurement of Thickness of Immobilized Layer on Immobilized Surface

The thickness of the immobilized layer on each PVC material surface was evaluated by a spectroscopic ellipsometer at an incident angle of 70° with a wavelength (A) of 400 to 700 nm.

As shown in Table 1 below, the coating thickness on the PVC surface after HT immobilization was 4.05 nm. It was shown that the thickness of the HT/Cu coating increased gradually as the feeding concentration of CUSO₄ increased, and that the coating thicknesses of HT/Cu0.25, HT/Cu0.5 and HT/Cu1 were 5.38, 6.47 and 8.07 nm, respectively.

TABLE 1
Changes in surface thickness by introduction
of heparin and copper ions

| Sample | Thickness (nm) | n (at 633 m) |
|---|---|---|
| HT | 4.05 | 1.3137 |
| HT/Cu0.25 | 5.38 | 1.2688 |
| HT/Cu0.5 | 6.47 | 1.3110 |
| HT/Cu1 | 8.07 | 1.3328 |

2-5: Evaluation of Chemical Composition of Immobilized Surface

The surface chemical composition of copper ions and the oxidation step were evaluated by XPS.

As shown in FIG. 3d, the presence of the Sap peak (166 eV) and the N1s peak (397 eV) in the spectra of HT and HT/Cu compared to that of the bare PVC indicates successful immobilization of the HT conjugate. In addition, the spectrum of HT/Cu showed the Cu₂p peak at 931 eV, indicating deposition of copper nanoparticles on the surface. The atomic percentages of these elements are shown in Table 2 below.

TABLE 2
Changes in surface components by introduction
of heparin and copper ions

| Sample | S (%) | Cl (%) | C (%) | N (%) | O (%) | Cu (%) |
|---|---|---|---|---|---|---|
| Bare | 0.06 | 14.75 | 75.38 | 1.68 | 8.1 | 0 |
| HT | 0.23 | 4.13 | 73.61 | 3.73 | 17.91 | 0.26 |
| HT/Cu | 1 | 4.32 | 69.53 | 3.6 | 19.6 | 1.51 |

The high resolution of the Cu₂p peak in the spectrum of HT/Cu showed that Cu₂p was split into Cu₂p3/2 (933.6 eV) and Cu₂p1/2 (952.38 eV) peaks, indicating the presence of Cu(II) and Cu(I) (FIG. 3e). Thereby, the redox-chelation reaction between Cu(II) and the catechol group was confirmed. The above results mean that heparin and copper nanoparticles were successfully co-immobilized on the PVC surface by the “one-pot” tyrosinase (Tyr)-mediated reaction.

2-6: Evaluation of In Vitro Release of Nitric Oxide

The release of nitric oxide generated by copper particles was evaluated using Saville-Griess reagent.

Copper ions can catalyze the decomposition of RSNO to generate NO in the presence of a reducing agent such as glutathione (GSH). In the present disclosure, GSNO was selected as a source that generates NO in the presence of GSH, and the reaction mechanism that generates NO is as follows:

GSNO+2OH⁻→GS⁻+NO₂⁻+H₂O

2Cu²⁺+2GS⁻→2Cu⁺+GSSG

2Cu⁺+GSNO→Cu²⁺+GS⁻+NO

Specifically, each PVC surface after immobilization was incubated with gentle shaking in 1 ml of a solution containing 100 μM of GNSO and 100 μM of glutathione (GSH) at 37° C. in a dark place. At a predetermined time point, the extracted solution was collected and allowed to react with the same volume of Griess solution (40 g/L) at room temperature for 15 minutes. The absorbance of the reaction solution was measured at 540 nm using a microreader plate. With reference of the standard curve with the known NO concentration (0 to 1 μM), the cumulative concentration of generated nitric oxide was calculated.

As shown in FIG. 4a, in the absence of the catalytic activity of copper ions, the HT surface released about 8 μM of NO after 14 days due to slow decomposition of GSNO under physiological conditions, whereas the HT/Cu surfaces significantly increased the NO release rate in the presence of GSNO. The release profile increased rapidly during the first 5 days and was maintained for 14 days. The release of NO was proportional to the feeding concentration of CuSO₄ used for immobilization. Specifically, it was confirmed that the cumulative release of NO after 14 days was 80 μM for HT/Cu1, 58 μM for HT/Cu0.5, and 32 μM for HT/Cu0.25.

2-7: Evaluation of In Vitro Copper Release

Copper release from each immobilized surface was detected by inductively coupled plasma-optical emission spectroscopy (ICP-OES). To obtain a leachate, each immobilized PVC substrate was incubated in 1 ml of PBS in an incubator at 37° C. At a predetermined time point, the leachate was collected, and the PBS was replaced with a fresh PBS solution.

As shown in FIG. 4b, it was confirmed that all the HT/Cu surfaces showed a high release rate for the first 7 days, and then showed a low release rate, and were stably maintained even after they were immersed in PBS for 7 days.

As the concentration of CuSO₄ increased, the amount of copper nanoparticles immobilized increased and the amount of Cu ions released increased. HT/Cu1 showed the largest amount of copper release (80 ppb), but this amount is a concentration lower than the concentration (1,000 ppb) that can cause cytotoxicity.

### [Example 3] Surface Immobilization Using Calcium Peroxide and Polyphenol Oxidase

In the present disclosure, the catalytic reaction between polyphenol oxidase and calcium peroxide was used to more effectively immobilize heparin on a surface.

A PTFE surface (13 mm diameter) was immobilized in a solution containing HT (0.5 wt %), Tyr (0.1 kU/ml) and CaO₂ (0 to 0.1 wt %) at 37° C. for each of 1, 2 and 4 hours. All the reactants were dissolved in Tris buffer (0.01 M, pH 8.2). Then, the immobilized surfaces were sonicated for 5 minutes, washed with 1 wt % of SDS for 1 hour, and then washed three times with distilled water to remove all unreacted reactants. The produced samples were dried with nitrogen gas to obtain immobilized surfaces.

TABLE 3
Composition ratio depending on calcium peroxide
concentration and reaction time

| | HT | CaO₂ | Tyr | |
|---|---|---|---|---|
| | concen- | concen- | concen- | Reaction |
| | tration | tration | tration | time |
| Sample | (wt %) | (wt %) | (kU/mL) | (h) |
| HT/C0 | 0.5 | 0 | 0.1 | 1, 2 |
| HT/C0.025 | 0.5 | 0.025 | 0.1 | or 4 |
| HT/C0.05 | 0.5 | 0.05 | 0.1 | |
| HT/C0.1 | 0.5 | 0.1 | 0.1 | |

The amount of heparin immobilized in the same manner as in Example 2-1. As a result, as shown in FIG. 6, it was confirmed that, as the reaction time increased and the CaO₂ concentration increased, the amount of heparin immobilized increased.

In addition, the water contact angle of each surface was analyzed in the same manner as in Example 2-3. As a result, as shown in FIG. 7, it was confirmed that, as the reaction time increased, the amount of heparin immobilized on the surface increased, so that the water contact angle decreased.

### Example 4

Analysis of Inhibition of Platelet Adhesion and Activation on Surface Having Heparin and Copper Nanoparticles Immobilized Thereon

4-1: Platelet Adhesion and Activation on Surface

In order to evaluation the inhibition of platelet adhesion and activation, each immobilized surface was analyzed by FE-SEM. Platelet-rich plasma (PRP) was isolated by centrifuging rat whole blood at 1,500 rpm for 15 minutes. After sterilization, each surface was incubated with 500 μl of PRP at 37° C. for 30 minutes in the presence or absence of a nitric oxide donor. After washing with DPBS, each surface was fixed using a 4% para-formaldehyde solution for 12 hours. Before FE-SEM, each sample was dewatered, dealcoholized and free-dried, followed by FE-SEM analysis.

As shown in FIG. Ba, Platelets attached to the bare PVC showed a dendritic or diffused dendritic form at high density. Meanwhile, the HT and HT/Cu surfaces significantly reduced platelet adhesion regardless of the presence of the NO donor, indicating that the antiplatelet activity of heparin was preserved. In addition, it was confirmed that the HT/Cu surface showed the lowest platelet adhesion and activation in the presence of the NO donor, and this result is due to the synergistic effect of heparin and the in situ generation of NO from the surface.

4-2: Evaluation of Level of Cyclic GMP in Cells Synthesized by Platelets

In healthy blood vessels, endothelial cells release NO, stimulate the activity of soluble guanylate cyclase which is an NO acceptor, increase the level of cGMP that controls platelet inhibition, and also induce smooth muscle cell relaxation. Thus, in the present disclosure, the degree of cGMP synthesis in platelets incubated with each of a bare surface and immobilized surfaces was evaluated.

Specifically, each surface was incubated in 500 μl of PRP at 37° C. for 30 minutes in the presence or absence of a nitric oxide donor, and then cGMP assay was performed. 100 μl of 10% Triton X-100 was added thereto, and the cell membrane was lysed by sonication. Then, the resulting solution was centrifuged at 3,000 rpm for 5 minutes, and the supernatant was collected. The cGMP level was measured using a Cyclic GMP Complete ELISA Kit (Abcam, Dawinbio Inc., Korea).

As shown in FIG. 8b, it was confirmed that the HT/Cu surfaces significantly promoted cGMP synthesis in the presence of GSNO, compared to the bare PVC surface and the HT surface. In addition, the level of cGMP synthesized from the platelets increased as the concentration of CuSO₄ increased.

These result mean that the HT/Cu surfaces exhibit the effect of improving plasma formation by producing NO from GSNO and inhibiting the adhesion and activation of platelets

### Example 5

Evaluation of Adhesion, Proliferation and Migration of Endothelial Cells on Surface Having Heparin and Copper Nanoparticles Immobilized Thereon

5-1: Evaluation of Adhesion and Proliferation of Endothelial Cells

In the present disclosure, the adhesion and proliferation of endothelial cells on the HT/Cu surfaces, the HT surface and the bare PVC surface were examined.

Specifically, HUVECs (1×10⁴ cells/ml) on each sterilized immobilized surface were incubated in a CO₂ incubator with an EBM-2 medium containing EGM-2 and 1% PS. After 24 hours and 72 hours of incubation, each surface was washed with DPBS to remove non-adherent cells.

Adhesion and proliferation of HUVECs was evaluated by WST-1 assay. The cells attached to the surface were incubated with 1 ml of a medium containing a 10% WST-1 reagent at 37° C. for 1 hour. After incubation, the absorbance at 450 nm was measured.

In addition, the cells attached to the surface were fixed with 4% para-formaldehyde at 37° C. for 4 hours. Then, the fixed cells were incubated with a rhodamine solution (10 μg/ml) at 37° C. for 30 minutes. Then, the morphology of the cells was analyzed by a fluorescence microscope.

As shown in FIGS. 9a and 9b, it was confirmed that the viability of HUVECs was significantly higher on the immobilized surfaces than the bare PVC surface. In particular, it was confirmed that, as the amount of CuSO₄ used in the immobilization step in the presence of the NO donor increased, the viability of HUVECs was higher. In addition, it was confirmed that the HUVECs incubated on the HT/Cu surface spread more than the cells incubated on the bare PVC and HT surfaces.

5-2: Evaluation of Migration of Endothelial Cells

In order to evaluate the effect of generated NO on the promotion of endothelial cell proliferation, the migration rate of HUVECs was analyzed using scratch assay.

A monolayer of endothelial cells on each immobilized surface was scratched in a straight line using a pipette tip to create similar sized scratches. The scratched surface was washed with an incubation medium. After 0, 4, 10 and 22 hours of incubation, the image of the scratch was captured and the migration rate of endothelial cells was observed. The image was analyzed with Image J software, and the percentage of the area covered by the migrated cells was calculated.

As shown in FIG. 9c, it was confirmed that the migration rate of the cells incubated with the immobilized surface was higher than the migration rate of the cells incubated with the bare PVC surface, and that the cell migration on the HT/Cu surface in the presence of the NO donor significantly increased. In addition, as shown in FIG. 9d, it was confirmed that the HUVECs covered about 55% and 80% of the scratch area after 4 hours and 10 hours, respectively. The HT-immobilized surface showed a lower scratch cover rate than the HT/Cu surface, but promoted cell migration, so that all scratched portions were covered after 22 hours.

From the above results, it was confirmed that the HT/Cu surface promoted NO release and significantly promoted endothelial cell proliferation.

### Example 6

### Evaluation of Adhesion and Proliferation of Smooth Muscle Cells on Surface Having Heparin and Copper Nanoparticles Immobilized Thereon

Adhesion and proliferation of human umbilical artery smooth muscle cells (HUASMCs) on each surface having heparin and copper nanoparticles immobilized thereon were evaluated by WST-1 assay and rhodamine staining assay. HUASMCs (1×10⁴ cells/ml) were incubated on each surface in the presence or absence of a nitric oxide donor. After 24 hours and 72 hours of incubation, WST-1 assay and rhodamine staining assay were performed according to the same procedure as described above with respect to “Evaluation of Adhesion and Proliferation of Endothelial Cells”.

As shown in FIG. 10, it was confirmed that, in the absence of the NO donor, there was no significant difference in cell viability between the immobilized surface and the bare surface, but in the presence of the NO donor, the HT/Cu surface showed a decrease in the viability of HUASMCs and distinct shrinkage compared to the bare surface and the HT surface.

These results indicate that the HT/Cu surface selectively exhibits promoting activity and inhibitory activity on endothelial cells and smooth muscle cells, respectively.

### Example 7

Production of Surface Having Heparin and NO-Generating Catalyst Immobilized Thereon, Depending on Kind of Material Surface

7-1: Production of Immobilized Surface and Surface Analysis

In the present disclosure, analysis was performed on whether heparin and a NO-generating catalyst are effectively co-immobilized regardless of the kind of material surface.

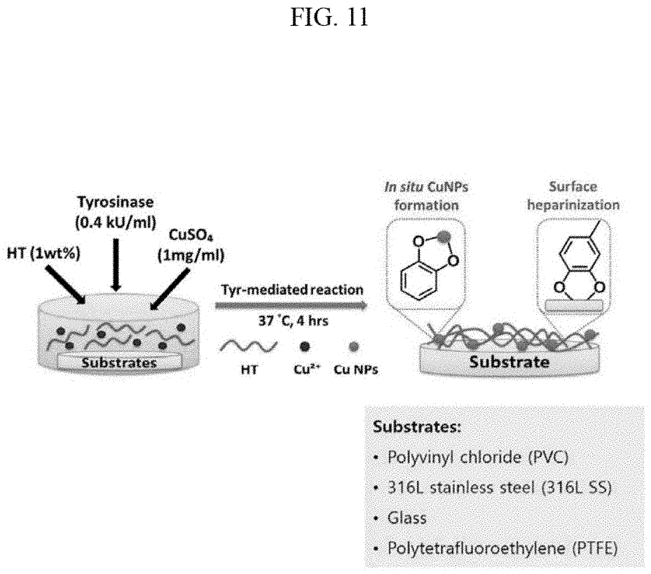

As material surfaces, polyvinyl chloride (PVC), 316L stainless steel (316L SS), glass, and polytetrafluoroethylene (PTFE) were used. According to the method described in Example 3 and FIG. 11, heparin and copper particles were immobilized on each material surface. Thereafter, the amount of heparin immobilized on each surface was analyzed using the method of Example 2-1, and the degree of release of NO was analyzed using the method of Example 2-6.

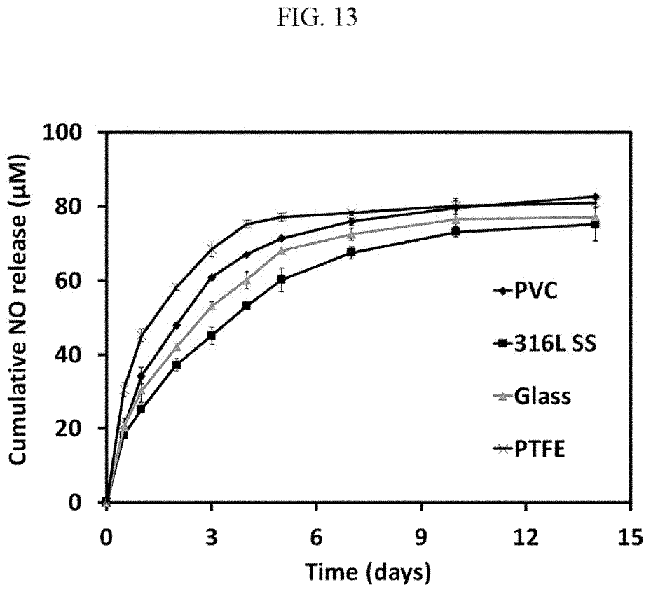

As a result, as shown in FIGS. 12 and 13, it was confirmed that, when PTFE was used as the material surface, the amount of heparin immobilized and the amount of NO released were somewhat higher. However, no difference depending on the kind of material surface was observed.

7-2: Evaluation of Adhesion and Proliferation of Endothelial Cells and Smooth Muscle Cells Depending on Kind of Immobilized Surface

The degrees of adhesion and proliferation of endothelial cells and smooth muscle cells on the PVC, 316L SS, glass and PTFE surfaces having heparin and copper particles immobilized thereon, produced in Example 7-1, were examined in the same manner as in Examples 5 and 6.

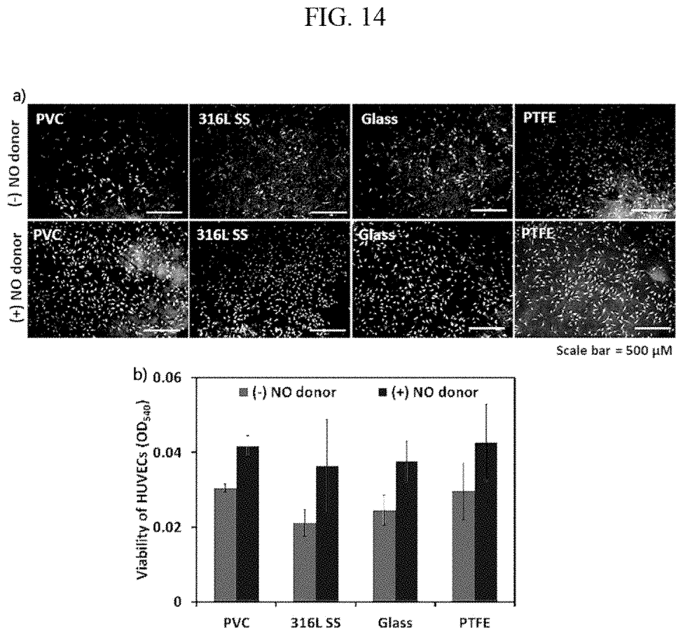

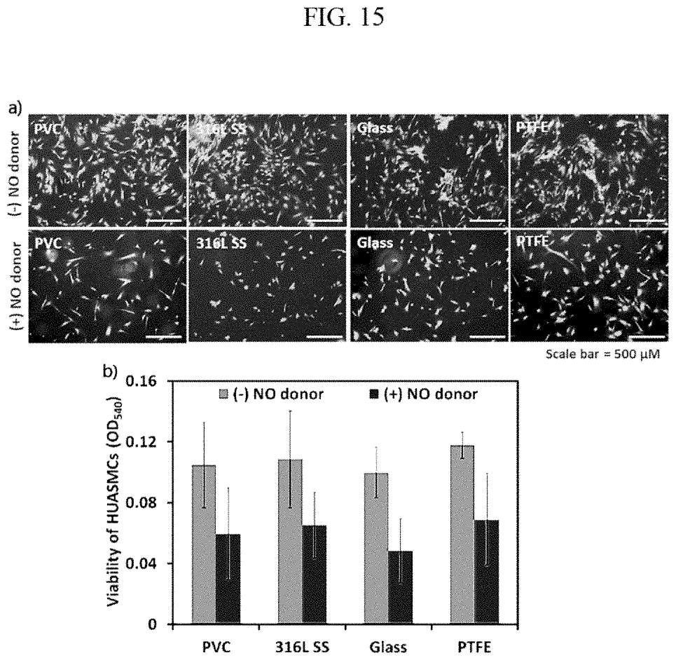

As a result, as shown in FIG. 14, the viability of the HUVECs was significantly enhanced regardless of the kind of material surface. In particular, it was confirmed that the viability of the HUVECs was higher in the presence of the NO donor. In addition, as shown in FIG. 15, it was confirmed that the adhesion and proliferation of the smooth muscle cells were significantly inhibited regardless of the kind of material surface.

That is, it was confirmed that the method of the present disclosure efficiently immobilized heparin and the NO-generating catalyst on the surfaces regardless of the kind of material surface, and also promoted the proliferation of endothelial cells while significantly inhibiting the adhesion and proliferation of smooth muscle cells.

As described above, it has been confirmed that the surface having heparin and the NO-generating catalyst co-immobilized thereon by the method of the present disclosure has high in vivo stability, continuously generates NO, and also promotes the proliferation of endothelial cells while significantly inhibiting the adhesion and activation of platelets and smooth muscle cells. Thus, the present disclosure may be advantageously applied to cardiovascular devices for inhibiting thrombosis and restenosis.

What is claimed is:

1. A method for co-immobilizing heparin and a NO-generating catalyst, the method comprising steps of:
(a) producing a heparin-phenol derivative by reacting a functional group-activated heparin with a phenol derivative; and
(b) treating a surface of a material with a mixture solution containing the heparin-phenol derivative, polyphenol oxidase and a NO-generating catalyst.

2. The method of claim 1, wherein the phenol derivative is one or more selected from the group consisting of tyramine, tyrosine hydroxyphenyl propionic acid, and 4-hydroxyphenyl acetic acid.

3. The method of claim 1, wherein the functional group-activated heparin in step (a) is a carboxyl group-activated heparin, a hydroxyl group-activated heparin, or an aldehyde group-activated heparin.

4. The method of claim 1, wherein the polyphenol oxidase in step (b) is tyrosinase.

5. The method of claim 1, wherein the mixture solution in step (b) contains 0.5 to 1 wt % of the heparin-phenol derivative, 0.1 to 0.4 kU/ml of the polyphenol oxidase, and 0.2 to 1 mg/ml of the NO-generating catalyst.

6. The method of claim 1, wherein the mixture solution in step (b) further contains calcium peroxide.

7. The method of claim 1, wherein the material in step (b) is a biocompatible material selected from among a biodegradable polymer, a non-degradable polymer, a metal and a ceramic material.

8. The method of claim 7, wherein the biodegradable polymer is one or more selected from the group consisting of polyglycolic acid, polylactide, poly(ε-caprolactone), poly(lactide-co-glycolide), poly(lactide-co-ε-caprolactone), poly(ε-caprolactone-co-glycolide), poly(ε-caprolactone-co-glycolide-co-lactide), polyhydroxybutyrate valerate (PHBV), polyorthoester (POE), polyethyleneoxide/polybutylene terephthalate (PEO/PBTP), chitosan, and collagen;
the non-degradable polymer is one or more selected from the group consisting of polyvinyl chloride, polyurethane (PUR), polyethylene terephthalate (PETP), polyamides, polyester, polyester elastomers, polymethylmethacrylate, polyolefins, silicone, polytetrafluoroethylene (PTFE), and polyfluorocarbon;
the metal is one or more selected from the group consisting of stainless steel, cobalt, chromium, titanium, nitinol, tantalum, and alloys thereof; and
the ceramic material is one or more selected from the group consisting of calcium phosphate, alumina, zirconia, silicon nitride, crystallized glass, and carbon materials.

9. A material for inhibiting thrombosis or restenosis, the material having a surface having heparin and a NO-generating catalyst co-immobilized thereon by the method of claim 1.

10. A cardiovascular device for inhibiting thrombosis or restenosis, the cardiovascular device comprising a material having a surface having heparin and a NO-generating catalyst co-immobilized thereon by the method of claim 1.

11. The cardiovascular device of claim 10, which is a stent.
